(19) United States
(12) Patent Application Publication
(10) Pub. No.: US 2005/0283165 A1
(43) Pub. Date: Dec. 22, 2005

(54) Hemostasis valve and method for assembling same

(76) Inventor: Donald L. Gadberry, San Juan Capistrano, CA (US)

Correspondence Address: APPLIED MEDICAL RESOUCES CORPORATION, 22872 Avenida Empresa, Rancho Santa Margarita, CA, 92688, US

(21) Appl. No.: 10/870,393

(22) Filed: Jun. 17, 2004

(51) Int. Cl. A61F 11/00

(52) U.S. Cl. 606/108

(57) ABSTRACT

The invention is directed to a sealing apparatus for sealing around a medical instrument, the sealing apparatus comprising a housing body; a two-piece cam body disposed inside the housing body; a sealing member disposed inside the cam body and having a channel extending axially of the cam body, the sealing member having at least two protrusions disposed around the channel; a plurality of pins operable to engage with the protrusions of the sealing member for movement relative to the cam body to radially compress the sealing member against and form a seal around the medical instrument; and biasing means for compressing the sealing member. In one aspect of the invention, the biasing means includes a first finger tab fixed to and extending laterally from the housing body, a second finger tab operatively associated with the cam body for movement relative to the housing body, the second finger tab extending laterally from the housing body in apposition to the first finger tab, and a spring being disposed between the first and second finger tabs to normally force the finger tabs apart. In another aspect, the protrusions in the cam body are more than two in number and are disposed around the channel at substantially equally spaced angular intervals. In another aspect, at least one of a radius, width, length, and angle of engagement of the pins may be varied or changed to exert different pressure against the surface of the protrusions. In another aspect, a process of assembling the sealing apparatus of the invention is disclosed, the process comprising the steps of placing the pins onto or against the protrusions of the sealing member; placing a first cam body half and a second cam body half over the sealing member and joining the two cam body halves together forming the 2-piece cam body; placing the housing body over the cam body; and placing the biasing means over the cam body inside the housing body.

### BACKGROUND OF THE INVENTION

1. Field of the Invention

This invention generally relates to access devices and, more specifically, to seals and other closure mechanisms applicable to devices.

2. Discussion of the Relevant Art

Access devices are commonly used in surgery to facilitate the introduction of various surgical instruments into vessels, conduits, cavities and other interior regions of the body. These access devices include, for example, seals which facilitate passing of an instrument such as a balloon catheter into a vessel, and trocars which facilitate the introduction of laparoscopic instruments into the abdomen of the body. Seals that operate to pass instruments into the vascular system must also operate as hemostasis valves that prevent blood loss during surgery. These seals may also be introduced into regions under pressure that may cause backflow bleeding if they are not properly controlled.

It is thus desirable to provide for an access device or seal that can facilitate the passing of a surgical instrument into the vascular system without the loss of blood. In addition, it is desirable that the access device or seal can provide injection of a fluid such as saline through an additional port without leaking. Stated another way, it is desirable to provide a hemostasis valve that operates like a door into the vascular system, the valve being manually opened or closed to regulate the force being exerted on an instrument being introduced therethrough. An advantage of such a valve is the instrument can be freely slid in or out of the valve while maintaining a seal around the instrument. The method of assembling the valve and seal should also be provided, and the seal should also be capable of conforming to objects of irregular shape which functions to axially retain an instrument extended therethrough.

### SUMMARY OF THE INVENTION

The invention is directed to a sealing apparatus for sealing around a medical instrument, the sealing apparatus comprising a housing body; a two-piece cam body disposed inside of the housing body; a sealing member disposed inside the cam body and having a channel extending axially of the cam body, the sealing member having at least two protrusions disposed around the channel; a plurality of pins operable to engage with the protrusions of the sealing member for movement relative to the cam body to radially compress the sealing member against and form a seal around the medical instrument; and biasing means for compressing the sealing member. In one aspect of the invention, the biasing means includes a first finger tab fixed to and extending laterally from the housing body, a second finger tab operatively associated with the cam body for movement relative to the housing body, the second finger tab extending laterally from the housing body in apposition to the first finger tab, and a spring being disposed between the first and second finger tabs to normally force the finger tabs apart.

In another aspect of the invention, the protrusions in the cam body are two or more in number and are disposed around the channel in the sealing member at substantially equally spaced angular intervals. For example, the protrusions may be four in number and angularly spaced at 90 degree intervals around the sealing member. The spring may be a torsion spring disposed over the cam body or a compression spring disposed between the first and second finger tabs. The sealing apparatus may further comprise a cap adapter operably attached at a distal end of the cam body, and an end cap operably attached at a proximal end of the cam body. In another aspect of the invention, the pins may be variably shaped so as to exert different pressure along the surface of the protrusions. In particular, at least one of a radius, width, length, and angle of engagement of the pins may be varied to exert different pressure against the surface of the protrusions. An advantage of this aspect of the invention is less spring force is required to completely seal the sealing member at the end of a closing stroke.

In another embodiment of the invention, a process of assembling the sealing apparatus of the invention is disclosed, the process comprising the steps of placing the pins onto or against the protrusions of the sealing member; placing a first cam body half and a second cam body half over the sealing member and joining the two cam body halves together forming the 2-piece cam body; placing the housing body over the cam body; and placing the biasing means over the cam body inside the housing body. The process may further comprise the step of squeezing two legs of the spring together and placing the coil portion over the cam body, attaching the cap adapter at the distal end of the cam body, and attaching the end cap at the proximal end of the cam body. The end cap may further comprise a tab for aligning with a slot in the housing body.

### DESCRIPTION OF THE DRAWINGS

FIG. 1 is a perspective view of a sealing apparatus in accordance with a first embodiment of the invention;

FIG. 2 is a cross-sectional view taken on the plane designated by line 2-2 of FIG. 1, showing the sealing apparatus with the sealing member in the normally closed condition;

FIG. 3 is an exploded perspective view of the sealing apparatus of the invention;

FIG. 4 is a cross-sectional view taken on the plane designated by line 3-3 of FIG. 2 showing the sealing apparatus with the sealing member in the opened condition; and

FIG. 5 is a cross-sectional view of a sealing apparatus having a different sealing member and pins in accordance with another embodiment of the invention.

### DESCRIPTION OF THE INVENTION

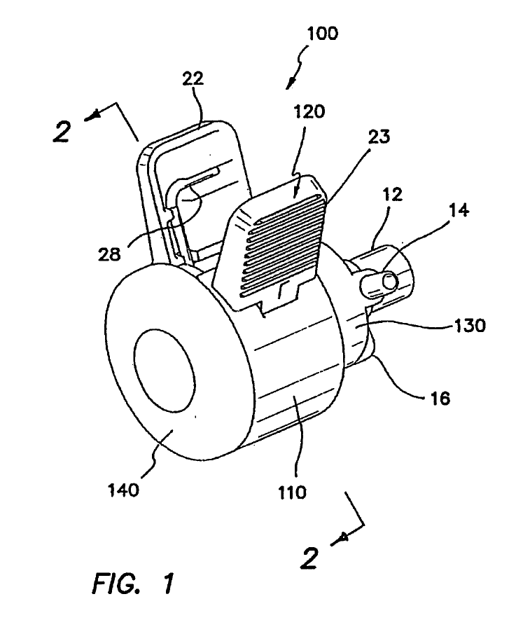

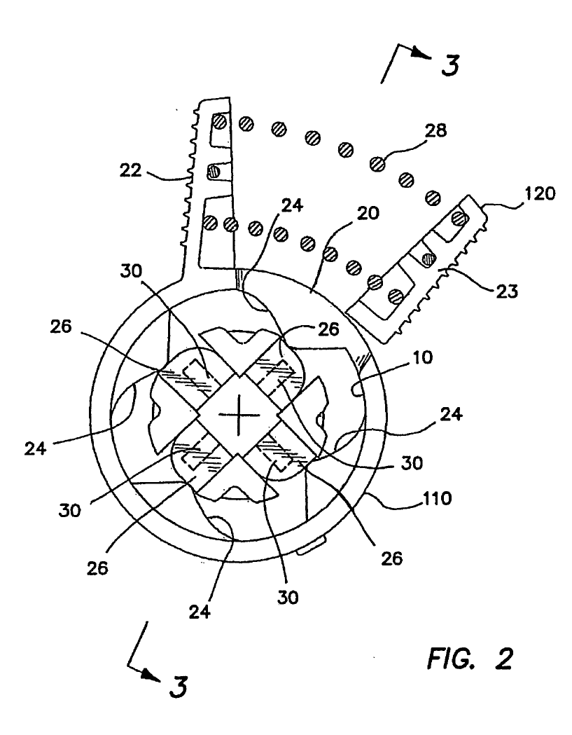

Referring to FIG. 1, there is shown a sealing apparatus 100 in accordance with a first embodiment of the invention comprising a housing body 110 including a stationary finger tab 22, a 2-piece cam body 120, a cap adapter 130 and an end cap 140. The housing body 110 includes an enlarged open-ended cavity 10 (see FIG. 2) and the cap adapter 130 includes a tubular conduit fitting 12 communicating with the cavity 10. In one embodiment, the cap adapter 130 further includes a side port 14 for communicating with the interior of the fitting 12. The cap adapter 130 may include a suture loop 16 that is formed integrally with the cap adapter 130. Referring to FIG. 2, the housing body 110 further includes an elongate arcuate slot 20 extending through the housing body 110 to accommodate a finger tab 23 of the cam body 120 in apposition to the stationary finger tab 22. The slot 20 opens through one end of the housing body 110 to facilitate assembly of the apparatus 100 as further discussed below.

In the assembled condition, the cam body 120 is concentrically received within the housing body 110, with the finger tab 23 extending through the slot 20. The exterior surfaces of the cam body 120 are of an arcuate configuration and configured to slide on the interior surface of the cavity 10. Interiorally, the housing body 110 is formed with four cam surfaces 24. The cam surfaces 24 are associated with a plurality of pawls or pins 26 which are spaced relative to one another. In one embodiment, counterclockwise movement of the cam body 120 relative to the housing body 110 functions to force the pins 26 inwardly, and clockwise movement functions to release the pins outwardly. A compression coil spring 28 is engaged between the finger tabs 22 and 23 to normally bias the finger tabs 22, 23 in an opposite direction to each other, forcing the pins 26 inward as shown in FIG. 2.

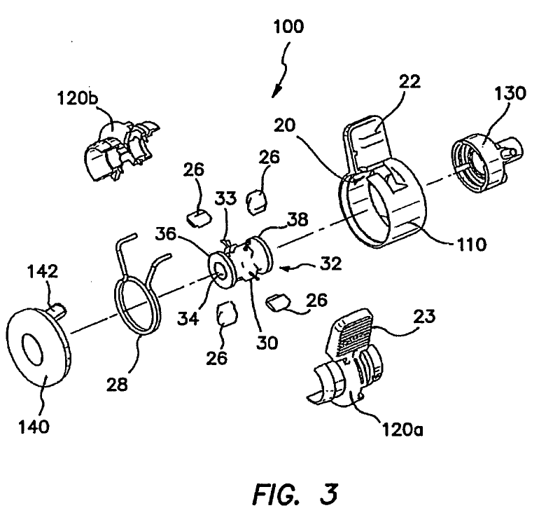

Referring to FIG. 3, there is shown an exploded perspective view of the sealing apparatus 100 of the invention. As illustrated in FIG. 3, the pins 26 engage protrusions or lugs 30 formed on an elastomeric sealing member 32. This sealing member 32 includes a central section 33, which extends longitudinally of the sealing member 32 between a pair of annular end flanges 36 and 38. Portions of the central section 33 and the end flanges 36, 38 define a central bore or passage 34, which extends axially through the sealing member 32. It is appreciated that the bore 34 can accept instruments up to 60 Fr in diameter.

The protrusions or lugs 30 extend longitudinally of the sealing member 32 between the end flanges 36, 38. The sealing element 32 can have a plurality of the protrusions or lugs 30 angularly spaced around the bore 34. In one embodiment, the sealing member 32 includes four radially extending lugs 30 equally spaced at ninety-degree intervals around the bore 34. This provides the sealing member 32 with a central portion 33, which in radial cross-section, may appear to have the configuration of a cross.

The material associated with at least the central portion 33 of the sealing member 32 is of particular interest to the present invention. This material is preferably very soft and pliable so that a pressure applied to the sealing member 32 by the pins 26 acting on the lugs 30 will force the central section 33 into the regions defining the bore 34. With an appropriate material, this pressure on the sealing element 32 will force it into contact with any object, such as a catheter or surgical instrument, extending through the bore 34. In this manner, the sealing element 32 tends to form a seal with the exterior surface of any such object or objects.

As explained in U.S. Pat. No. 5,127,626, which is herein incorporated by reference, another fluid characteristic associated with the material of the sealing member 32 is the property it has for transferring substantially all of the applied force to the region defining the bore 34. Thus, a pressure applied by pins 26 to the lugs 30 is substantially the same pressure exerted by the portions defining the bore 34 against an object extending through the bore. With a material of low durometer, very little energy is lost in deforming the sealing member 32.

It should be noted that any material having a low durometer should be confined in order that a pressure applied to the material will force it into the desired region such as the bore 34. In the illustrated embodiment, the housing body 110, end cap 140, and cap adapter 130 provide the confining means and are configured to define a cavity similar in shape to the exterior surface of the sealing member 32. Then as the pins 26 are moved radially against the protrusions or lugs 30 of the sealing member 32, this pressure forces the material of the central portions 33 into the region of the bore 34.

The exploded perspective view of FIG. 3 further shows how the sealing apparatus 100 is to be assembled. First, the sealing member 32 is placed on an assembly fixture; the pins 26 are then placed onto the protrusions or lugs 33, it should be noted that the radii of the pins 26 should all face the same direction. Next, hold a first cam body half 120a by the finger tab 23 and place it over the sealing member 32, making sure that the radii of pins 26 are in the same relative position to the first cam body half 120a. Each pin 26 may include a raised rib that matches up with a mating slot in the first cam body half 120a. Next, place a second cam body half 120b over the sealing member 32 and pins 26 so as to join the two cam body halves 120a, 120b together by their locating posts and holes. The housing body 110 is then placed over the cam body halves 120a, 120b such that cam surfaces 24 fit against pins 26. During this step, the two cam body halves 120a, 120b should be lightly press into the housing body 110 while pushing inward on the one visible pin 26 until the pin 26 fits into the proper place and the cam body halves 120a, 120b are all the way inside of the housing body 110.

The cap adapter 130 may then be placed onto the protruding end of the two cam body halves 120a, 120b, aligning the key and slot before pressing it firmly to snap-fit it onto the cam body halves. A spring 28 is then placed over the two cam body halves 120a, 120b inside the housing body 110 by squeezing the two legs of the spring 28 together and placing the coil portion over the cam body halves 120a, 120b. The spring 28 may include bent ends that are placed into raised ribs in the inside cavity of each of finger tabs 22, 23. Next, the end cap 140 having a tab 142 is placed onto the two cam body halves 120a, 120b, aligning the tab 142 with slot 20 in the housing body 110. In another aspect, a compression spring may be used in place of the torsion spring 28. In particular, the compression spring is placed between finger tabs 22, 23.

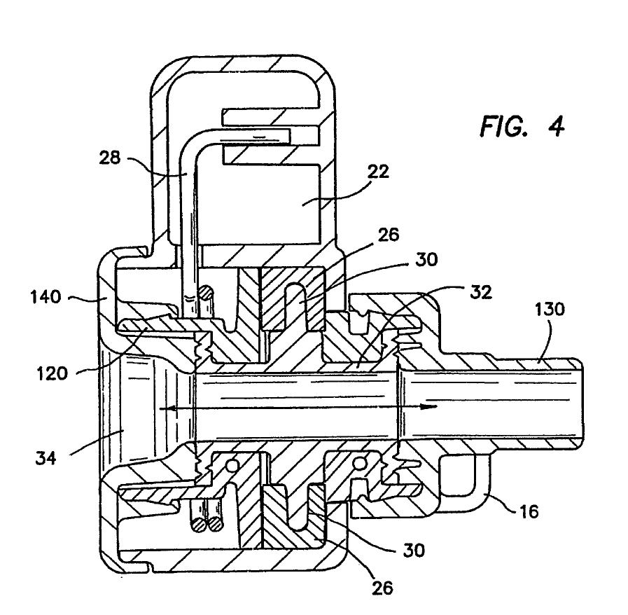

Once the sealing apparatus 100 is assembled, its operation is very simple. To condition the sealing apparatus 100 for passage of an instrument therethrough, it is simply necessary to squeeze the finger tabs 22, 23 toward each other, thus opening the bore or passage 34 as shown in FIG. 4 in a modulating fashion. The instrument may then be freely extended through the bore or passage 34 while controlling any leakage by means of applying more or less pressure on the finger tabs 22, 23. Upon being positioned as desired, the finger tabs 22, 23 are released, thus closing the passage into full sealed engagement with the instrument. The latter operation also functions to grip the instrument and hold it at the desired location. Release of the instrument is achieved by simply again squeezing the finger tabs 22, 23 together against the biasing force of the spring 28.

It is of advantage that the force applied to the protrusions or lugs 30 of the sealing member 32 be directed radially of the bore 34. This tends to insure that the material of the sealing member 32 is pushed most directly to fill the space of the bore 34. By guiding the pins 26 radially, the two cam body halves 120a, 120b provide means for translating the rotational movement of the cam surface 24 into a radial, linear movement of the pins 26.

It is appreciated that the force applied against the sealing member 32 should be directed from more than two radial directions. Only with the application of an applied force from multiple radial directions can one avoid the cat-eye leakage that is typical of the seals of the prior art at two opposing points along a traversing instrument or shaft. The configuration, however, may employ force from two radial directions in some applications. The conformity of the sealing member 32 to irregularly shaped objects extending through the bore 34 is greatly increased by the provision of, for example, four of the protrusions or lugs 30 in one embodiment. Particularly large diameters of the bore 34 may be accommodated with more than four of the protrusions or lugs 30. With a radial force applied along each of these lugs 30 the material tends to flow with little pressure into the region of the bore 34 thereby creating a compliant seal with any shaft or object extending through the bore 34.

As explained above, the compression spring 28 provides means for biasing the sealing member 32 against any object extending through the bore 34. If there is no such object, the spring will bias the sealing member 32 to entirely close the bore 34. Thus, the sealing member 32 can close to a diameter of zero (0) so as to form a seal against itself. An advantage associated with the present invention is it permits objects to be entirely removed from the bore 34 without permitting a loss of gas pressure or liquids that might otherwise pass through the unoccupied bore 34. At the same time, resistance applied against the compression spring 28 can be modulated to open the bore to significant diameters such as sixty French (60 Fr) to permit the insertion of objects with substantially no frictional resistance from the sealing member 32 while at the same time permitting little or no leakage back through the sealing member 32. Then by releasing pressure on the spring 28, the sealing member 32 can fully close against the exterior surface of the object to affect a tight seal of the bore 34 with the instrument inserted therein.

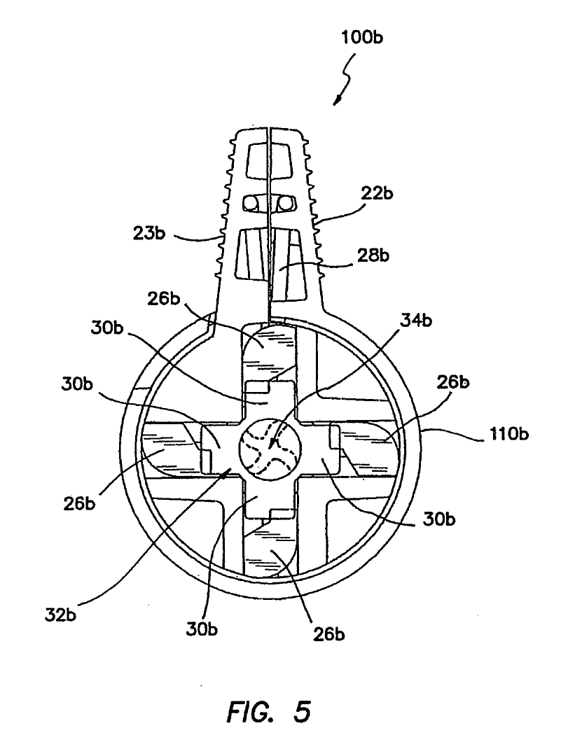

Referring to FIG. 5, there is shown a cross-sectional view of a sealing element 32b and pins 26b in accordance with another embodiment of the invention. The sealing element 32b includes a plurality of protrusions or lugs 30b for engaging pins 26b and defines a central bore or passage 34b that extends axially through the sealing member 32b. As illustrated in FIG. 5, the pins 26b are shaped such that a pressure applied to the pins 26b is unevenly exerted onto the lugs 30b so as to cause the bore 34b to close around an instrument faster with less spring force. An advantage of this aspect of the invention is less spring force is required at the end of a closing stroke to completely seal the center orifice. It is appreciated that pins 26b may have various shapes and sizes including radii, widths, etc., and angles of engagement with protrusions or lugs 30b so as to achieve the desired seal characteristics.

From the foregoing detailed description, it will be apparent that the present invention provides an apparatus for sealing around elongate instruments of various sizes and shapes, and a method of assembling the apparatus. It should be appreciated, however, that many other modifications could be made to the various disclosed embodiments without departing from the spirit and scope of the invention. For these reasons, the above description should not be construed as limiting the invention, but should be interpreted as merely exemplary of preferred embodiments.

1. An apparatus for sealing around a medical instrument, comprising:
a housing body 110;
a two-piece cam body 120 having a proximal end and a distal end, said cam body 120 disposed inside and extending axially of the housing body 110;
a sealing member 32 disposed inside the cam body 120 and having a channel extending axially of the cam body 120, said sealing member 32 having at least two protrusions 30 disposed around the channel;
a plurality of pins 26 operable to engage with the protrusions 30 of the sealing member 32 for movement relative to the cam body 120 to radially compress the sealing member 32 against and form a seal around the medical instrument; and
biasing means for compressing the sealing member 32.

2. The apparatus of claim 2, wherein the biasing means includes a first finger tab 22 fixed to and extending laterally from the housing body 110, a second finger tab 23 operatively associated with the cam body 120 for movement relative to the housing body 110, said second finger tab 23 extending laterally from the housing body 110 in apposition to the first finger tab 22, and a spring 28 disposed between the first and second finger tabs 22, 23 to normally force the finger tabs 22, 23 apart.

3. The apparatus of claim 1, wherein the protrusions 30 in the cam body 120 are more than two in number and are disposed around the channel in the sealing member 32 at substantially equally spaced angular intervals.

4. The apparatus of claim 1, wherein the protrusions 30 are four in number and angularly spaced at 90 degree intervals around the sealing member 32.

5. The apparatus of claim 2, wherein the spring 28 is a torsion spring disposed over the cam body 120.

6. The apparatus of claim 2, wherein the spring 28 is a compression spring.

7. The apparatus of claim 2, further comprising a cap adapter 130 operably attached at the distal end of the cam body 120.

8. The apparatus of claim 7, further comprising an end cap 140 operably attached at the proximal end of the cam body 120.

9. The apparatus of claim 1, wherein the pins 26 are variably shaped so as to exert different pressure along the surface of the protrusions 30.

10. The apparatus of claim 9, wherein at least one of a radius, width, length, and angle of engagement of the pins 26 is varied to exert different pressure against the surface of the protrusions 30.

11. A process of assembling an apparatus for sealing around a medical instrument, comprising the steps of:
placing a plurality of pins 26 onto or against protrusions 33 of a sealing member 32;
placing a first cam body half 120a and a second cam body half 120b over the sealing member 32 and joining the two cam body halves 120a, 120b together forming a 2-piece cam body 120, said cam body 120 having a proximal end and a distal end;
placing a housing body 110 over the cam body 120; and
placing a biasing means 28 over the cam body 120 inside the housing body 110.

12. The process of claim 11, wherein the biasing means 28 includes a first finger tab 22 fixed to and extending laterally from the housing body 110, a second finger tab 23 operatively associated with the cam body 120 for movement relative to the housing body 110, said second finger tab 23 extending laterally from the housing body 110 in apposition to the first finger tab 22, and a spring 28 having two legs disposed between the first and second finger tabs 22, 23 to normally force the finger tabs 22, 23 apart.

13. The process of claim 12, further comprising the step of squeezing the two legs of the spring 28 together and placing a coil portion of the spring 28 over the cam body 120.

14. The process of claim 13, further comprising the step of attaching a cap adapter 130 at the distal end of the cam body 120.

15. The process of claim 14, further comprising the step of attaching an end cap 140 at the proximal end of the cam body 120.

16. The process of claim 11, wherein the pins 26 are variably shaped so as to exert different pressure along the surface of the protrusions 30.

17. The process of claim 16, wherein at least one of a radius, width, length, and angle of engagement of the pins 26 is varied to exert different pressure against the surface of the protrusions 30.

18. The process of claim 15, wherein the end cap 140 further comprises a tab 142 for aligning with a slot 20 in the housing body 110.

19. An apparatus for sealing around a medical instrument, comprising:
a housing body 110;
a cam body 120 having a proximal end and a distal end, said cam body 120 disposed inside and extending axially of the housing body 110;
a sealing member 32 disposed inside the cam body 120 and having a channel extending axially of the cam body 120, said sealing member 32 having at least two protrusions 30 disposed around the channel at substantially equally spaced angular intervals;
a plurality of pins 26 operable to engage with the protrusions 30 of the sealing member 32 for movement relative to the cam body 120 to radially compress the sealing member 32 against and form a seal around the medical instrument; and
biasing means for compressing the sealing member 32,
wherein the pins 26 are variably shaped so as to exert different pressure along the surface of the protrusions 30.

20. The apparatus of claim 19, wherein at least one of a radius, width, length, and angle of engagement of the pins 26 is varied to exert different pressure against the surface of the protrusions 30.

21. The apparatus of claim 19, wherein the biasing means includes a first finger tab 22 fixed to and extending laterally from the housing body 110, a second finger tab 23 operatively associated with the cam body 120 for movement relative to the housing body 110, said second finger tab 23 extending laterally from the housing body 110 in apposition to the first finger tab 22, and a spring 28 disposed between the first and second finger tabs 22, 23 to normally force the finger tabs 22, 23 apart.

22. The apparatus of claim 19, wherein the protrusions 30 in the cam body 120 are more than two in number and are disposed around the channel in the sealing member 32 at substantially equally spaced angular intervals.

23. The apparatus of claim 19, wherein the protrusions 30 are four in number and angularly spaced at 90 degree intervals around the sealing member 32.

24. The apparatus of claim 21, wherein the spring 28 is a torsion spring disposed over the cam body 120.

25. The apparatus of claim 21, wherein the spring 28 is a compression spring.

26. The apparatus of claim 21, further comprising a cap adapter 130 operably attached at the distal end of the cam body 120.

27. The apparatus of claim 26, further comprising an end cap 140 operably attached at the proximal end of the cam body 120.
